(19) United States
(12) Patent Application Publication
(10) Pub. No.: US 2006/0270866 A1
(43) Pub. Date: Nov. 30, 2006

(54) Processes for production of esters from olefin-containing hydrocarbon streams and vegetable or animal oils

(76) Inventors: Richard Sapienza, East Moriches, NY (US); Axel Johnson, North Babylon, NY (US)

Correspondence Address: HEDMAN & COSTIGAN P.C., 1185 AVENUE OF THE AMERICAS, NEW YORK, NY, 10036, US

(21) Appl. No.: 11/439,899

(22) Filed: May 24, 2006

Related U.S. Application Data
(60) Provisional application No. 60/684,377, filed on May 25, 2005.

(51) Int. Cl. C07C 51/43

(52) U.S. Cl. 554/174

(57) ABSTRACT

The present invention provides processes for producing esters from olefin-containing hydrocarbon streams and vegetable or animal oils.

### CROSS REFERENCE TO RELATED APPLICATIONS

This application claims priority from U.S. Provisional Application Ser. No. 60/684,377, filed May 25, 2005.

### FIELD OF THE INVENTION

The present invention relates to processes for the production of monocarboxylic acid esters from reactions involving olefin-containing hydrocarbon streams and triglyceride-containing vegetable or animal oils. The esters so produced can be utilized as diesel fuels (biodiesel), fuel for heating systems, ecological solvents, basic compounds for production of sulfonates of fatty alcohols, amides, ester dimers, etc. More particularly, the present invention relates to the use of streams comprising olefins having 2 to 6 carbon atoms obtained from hydrocarbon catalytic cracking and thermal cracking processes employed, for example, in petroleum refining and petrochemical production.

Three of the objectives of the present invention are:
i. In the case of diesel fuel applications, to produce a product having a lower cloud point than is the case when biodiesel is produced using methanol,
ii. To avoid the possibility of environmental issues resulting from methanol release on biodegradation of biodiesel produced using methanol,
iii. To improve the economics of biodiesel production by: (1) utilizing lower cost raw materials (light olefins vs. methanol or ethanol) and (2) centralizing production in a refinery or petrochemical plant wherein one can achieve economies of scale together with use of common processing infrastructure, or a combination of (1) and (2).

### BACKGROUND AND SUMMARY OF THE INVENTION

Esters of fatty acids are becoming increasingly important as diesel fuels. In many European countries the inclusion of biodiesel in diesel fuel blend is mandated. In the United States, recent legislation has provided tax incentives for inclusion of biodiesel in diesel fuel blends. Most present and planned production is based upon production of the methyl ester using methanol as the reactant with the triglycerides contained in vegetable and animal oils.

The popularity of methyl esters in this role has generated the term “biodiesel” as referring to esters of fatty acids derived from fatty acid triglycerides contained in vegetable and/or animal oils. Alkyl esters, such as methyl esters, of fatty acids generally are preferred over vegetable oils or animal fats, for use as, or in blends of, diesel fuel because the alkyl esters have a viscosity compatible with diesel fuel specifications.

As sources of the fatty acid glycerides, all naturally occurring vegetable and animal fats and oils, all partly or fully synthetic fatty acid glycerides and used fatty acid glycerides, such as used frying oils and fats, as well as used industrial fats and oils based on glyceride, such as soybean oil, sunflower oil, linseed oil, rapeseed oil, castor oil, palm oil, palm kernel oil, coconut oil, cottonseed oil, peanut oil, olive oil, beef tallow, used frying oil or grease, used hydraulic or lubricating oils, can be considered. These oils may be used in the raw state from hot or cold pressing or from an extraction with free fatty acids or in an arbitrarily purified form. They can be present alone or in arbitrary mixture proportions with one another.

While biodiesel has several advantages, including a high cetane number and reduced particulate and sulfur emissions, fuels based on fatty acid methyl esters do have some adverse effects. In particular, saturated long-chain fatty acid esters that result from the esterification of the fatty acids with methanol or ethanol tend to freeze at higher temperatures than the typical components of petroleum derived diesel fuel. This results in higher cloud points—a measure of the low temperature performance of diesel fuel. Cloud point data are summarized in Table 1.

TABLE 1
Onset of Crystallization of Diesel Fuel and Various
Pure Esters of Natural Fats and Oils in ° C.

| | Diesel | | Low | | |
|---|---|---|---|---|---|
| Substance | Fuel | Soybean Oil | Palmitic Soy | Canola | Lard |
| Petroleum' | −11.9 | | | | |
| Methyl esters | | 5.2 | −1.0 | | |
| Ethyl esters | | 1.4 | −3.8 | 0.4 | 12.1 |
| Isopropyl esters | | −6.0 | −12.0 | −6.8 | 4.0 |
| 2-Butyl esters | | −9.0 | −18.5 | −8.1 | 0.5 |
| Tert-butyl esters | | −8.0 | | | |

These results reveal that the temperatures of crystallization onset of branched-chain isopropyl and 2-butyl soybean oil esters are substantially lower than the onset of crystallization of methyl and ethyl soybean oil esters. Specifically, 2-butyl ester has an onset of crystallization of about −9° C. to −20° C. The esters of canola oil performed comparably to soybean oil esters.

Most biodiesel production is based upon methanol as a reactant in the transesterification reaction with triglycerides. In addition to the cloud point issue discussed above, methanol is an expensive commodity typically produced from natural gas and other hydrocarbons. The other alcohol frequently discussed is ethanol—again, ethanol is a costly material

Further, as biodiesel fuels are becoming commercialized, their fate in the environment is an area of concern because petroleum spills constitute a major source of contamination of the ecosystem. Among these concerns, water quality is one of the most important issues for living systems. Biodiesel methyl esters readily are biodegraded under both oxic and anoxic conditions by the natural flora of soil and freshwater to the fatty acid and methanol. Although biodiesel is not water soluble, if these methyl esters enter the aquatic environment in the course of their use or disposal, their biodegradation could create a water pollution problem.

For these reasons, an economic source of reactive agent or agents to replace methanol in the production of esters from vegetable and animal oils, and agents producing biodiesel with lower cloud point are desirable objectives.

A further issue relates to the present development of the biodiesel industry in a dispersed or “stand-alone” manner. Typically, biodiesel facilities have been located near the source of the vegetable or animal oils. Methanol is produced at sources of hydrocarbons, such as natural gas, and then must be transported to the biodiesel production facility. In many cases the source of the methanol may be outside the U.S. The biodiesel product must usually then be sent to yet another location to be blended with petroleum base diesel. There would be considerable savings in transportation, storage and infrastructure, as well as substantial economies of scale, optimization by integration and potential for use of less costly raw materials if facilities could be located at existing hydrocarbon processing plants, such as petroleum refineries or chemical plants.

To address these issues, the present inventors surprisingly have found that novel combinations of existing and developmental technologies in the context of integration with petroleum refining and/or petrochemical operations can solve or address the issues outlined above and thereby solve a long felt need in the art. The inventors have discovered that the issues centered on the use of methanol as the reagent for production of esters from vegetable and animal oils, and the economic issues arising from the “stand-alone” nature of the developing industry, both can be addressed by substituting for methanol the olefins contained in one or more of various streams produced in the course of normal operations of petroleum refining and petrochemical processes.

### BRIEF DESCRIPTION OF THE DRAWINGS

FIG. 1 shows a flow chart of a process for esterification of fatty acids with olefins that may be employed in the practice of preferred embodiments of the present invention.

FIG. 2 shows a flow chart of a process for triglyceride hydrolysis by esterification with isobutylene that may be employed in the practice of preferred embodiments of the present invention.

FIG. 3 shows a flow chart of a process for olefin hydration to monoalcohols followed by transesterification of triglycerides that may be employed in the practice of preferred embodiments of the present invention.

FIG. 4 shows a flow chart of a process for integration of monoester production with alkylation that may be employed in the practice of preferred embodiments of the present invention.

### DETAILED DESCRIPTION OF THE PRESENT INVENTION

There are several extant technologies and new developments that promise to be useful in production of diesel fuel components from vegetable and animal fats and oils in the context of integration with petroleum processing. If one is to start with hydrocarbon derived olefins as a raw material to react with triglyceride-containing vegetable and animal products, it will be necessary to add three molecules of water for each molecule of triglyceride converted. This water can be introduced into the reaction sequence by either (1) converting the triglycerides to fatty acids and glycerin by hydrolysis or (2) by converting the olefins to alcohols—typically also by hydrolysis. Processes for these various reactions and for the conversion to monoesters are summarized below together with descriptions of petroleum refining and petrochemical processes relevant to the invention.

1. Triglyceride Hydrolysis

This reaction (fat splitting) commonly is used in modern0 processing for the manufacture of fatty acids for producing soap and other fatty acid derivatives. The fatty acids are produced by hydrolysis of triglycerides contained in vegetable or animal oils and/or fats. This hydration can be carried out with water alone or with an acid catalyst. In some cases this hydrolysis is conducted at high temperature and pressure (ca 500° F. and 600 psi) in a vertical counter flow reactor with the fat/oil phase flowing upward and the hot water/glycerol phase flowing downward. The water/glycerol stream will contain about 12-20% glycerol and some organic impurities dependent on the feed characteristics. This process can be practiced at the oil/fat production site (e.g., soybean processing facility or fat rendering plant) and the fatty acids then shipped to the point of monoester production. Alternatively, the oil/fat feed can be processed at the hydrocarbon processing facility (e.g., petroleum refinery or petrochemical processing plant).

2. Monoalcohol Production from Olefins

There are several commercial processes for converting monoolefins to monoalcohols. A common technique is by direct hydration of monoolefins employing a phosphoric acid on silica catalyst (see Ching, D. W., Process Technology Quarterly, January 1999). Other direct hydration processes employ ion exchange resin catalyst, such as, but not limited to, Rohm & Haas Amberlyst XE365. Another technique is indirect hydration with sulfuric acid as the catalyst. Any of the monoolefin to monoalcohol processes known to those skilled in the art may be employed in accordance with the practice of the present invention.

3. Esterification

The fatty acids recovered from hydrolysis of fats and oils can be converted to monoesters for use as biodiesel either by reaction with the selected monoacohols, or, as conceived by the inventors, by reaction with olefins produced in processing of hydrocarbons.

a. Reaction with Monoalcohols
Jeromin et al. (U.S. Pat. No. 4,698,186) teaches a process for reducing the free fatty acid content of fats and oils by reacting with a lower monoalcohol in the presence of an acidic ion exchange resin as a solid esterifications catalyst. This technique also could be applied to the larger scale conversion of separated fatty acids. While this could be a convenient means of shipping the basic components to a point of monoester production, the reaction is not very efficient on an overall basis as it requires two hydration processes—only one of which is necessary to satisfy the stoichiometry of monoester production from triglycerides and olefins. As a result water will be a product of the esterification reaction.

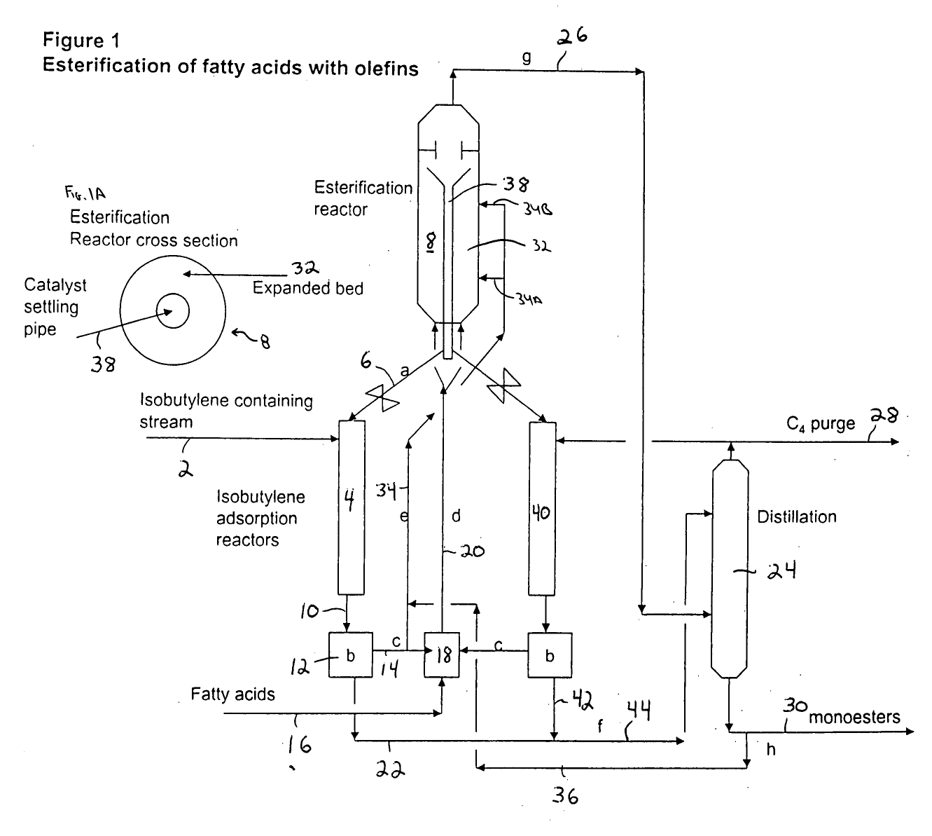

b. Fatty Acid/Olefin Reaction
The present inventors further have conceived of a means to avoid the excess hydration in utilizing olefins by employing direct reaction of the fatty acid with the olefin—most particularly with isobutylene. The reaction mechanism that the present inventors have conceived of has some similarity to the commonly practiced reaction of methanol with isobutylene to produce methyl tertiary butyl ether (MTBE). In that process a mixed C₄ stream—containing isomers of both butylene and butane—is employed together with methanol and reacted in the presence of an acidic ion exchange catalyst. The tertiary carbon in isobutylene reacts preferentially with the acidic site to form a carbonium ion which then reacts with the hydroxyl group of the methanol. Overall the MTBE reaction is:
   CH₃—OH+iC₄H₈→CH₃—O—C₄H₉
   The analogous monoester reaction substitutes a fatty acid for the methanol leading to:
   R—CO—OH+iC₄H₈→R—CO—O—C₄H₉
It may be possible to employ part or all of existing MTBE production facilities for conversion to this operation. There will be some tendency for production of di-isobutylene (DIB or isooctene) or higher oligomers. DIB will be separated in subsequent distillation and can be a useful gasoline component, and the higher oligomers can be left in the monoester stream as diesel components.
As one would expect, it is reported that the reaction rate of the ethanol/isobutylene reaction to form ETBE (ethyl tert butyl ether) is slower than the corresponding methanol reaction to form MTBE. It will be expected that the fatty acid/isobutylene reaction rate will be even slower. Under these circumstances, if the isobutylene concentration is maintained at a sufficient level to achieve a satisfactory reaction rate, the competing isobutylene oligomerization reactions may be favored. To solve this problem, the inventors have conceived of a system in which the two basic reactions (1) formation of a carbonium ion at the catalyst strong acid site, and (2) reaction of the fatty acid with the carbonium ion are conducted separately in a manner to optimize monoester production and minimize oligomerization. The essential features of the concept involve at least partially saturating the acid sites of the catalyst with isobutylene and then exposing said saturated catalyst to the fatty acids for reaction to form esters. This reaction sequence can be carried out in a single catalyst-containing reactor that alternately is fed with an isobutylene-containing stream and then with a fatty acid containing stream. This sequential process can be on a batch basis or on a continuous basis employing swing reactors in parallel. Alternatively, a continuous process employing catalyst movement between reactors can be employed as described below:
1. Catalyst, probably in a bead form, circulates between (1) an esterification reactor system and (2) an isobutylene adsorption system.
2. After the catalyst leaves the esterification system it is contacted with isobutylene under time and temperature conditions so as to achieve a significant level of saturation of the acid sites with isobutylene while minimizing oligomerization reactions. This reaction system is followed by a catalyst/hydrocarbon separation system—possibly a liquid cyclone.
3. The separated, isobutylene saturated catalyst is mixed with the fatty acid stream and reacted under conditions to achieve maximum conversion of the fatty acids. This system may involve more than one stage to achieve the desired result. Again, there is a catalyst separation system from which the product is separated from the catalyst and the catalyst is returned to step 2.
There are many possible configurations of process equipment known to those skilled in the art that can achieve the reaction sequence described above. One such configuration is illustrated in FIG. 1.
In the example of FIG. 1, an isobutylene-containing stream 2 is contacted in isobutylene adsorption reactor 4 with catalyst from a line 6 (a) withdrawn from the esterification reactor 8 and the mixture is fed to an isobutylene adsorption reactor 4. The isobutylene-containing stream 2 can be a butane-butylene (BB) stream from any of the sources described below (6a), or a stream containing isobutylene prepared from a BB stream or from butane by one of the processes known in the art. (An example is production of MTBE from a BB stream and then cracking of MTBE to produce isobutylene and methanol for recycle to the MTBE production step.) The isobutylene adsorption reactor 4 is configured to be an essentially plug flow reactor to avoid back-mixing in order to insure that the catalyst leaving has been appropriately contacted with isobutylene.

The effluent from the isobutylene adsorption reactors 4 is directed via a line 10 to a separator 12 and the isobutylene treated catalyst is removed from separator 12 in a line 14 for mixing with the fatty acid feed in a line 16 in a mixer 18. The mixed isobutylene treated catalyst and fatty acid feed are directed via a line 20 to the esterification reactor 8. The liquid separated in separator 12 in a stream 22 is principally unconverted BB plus some liquid having essentially the composition of the esterification reactor effluent carried in with the catalyst. This stream 22 is sent to distillation 24 for separation together with the esterification reactor effluent 26 into a C₄ purge overhead stream 28 and a monoester bottoms stream 30.
In the esterification reactor cross-section shown in FIG. 1A, the esterification reactor 8 comprises an expanded bed reactor 32 (around a catalyst settling pipe 38) sized so that the feed flow rate is sufficient to maintain the expanded state. Again, it is desirable that the liquid flow involve a minimum of back mixing. At the same time, co-current flow of catalyst and liquid will result in a situation where the later stage of the reaction—where the concentration of unconverted fatty acids is least—will also be the point where the catalyst has lost the bulk of its adsorbed isobutylene.
Accordingly, referring back to FIG. 1, it may be desirable to introduce freshly prepared catalyst from line 14 at several points along the reaction path via line 34 and 34A and 34B. A suitable carrier liquid for the catalyst could be a recycle stream 36 of monoester from monoester product stream 30. As the overall reaction is exothermic, the quantity and temperature of this recycle can also serve as a reactor temperature control mechanism.
At the top of the esterification reactor 8 there is a settling zone where catalyst settles into the central downcomer catalyst settling pipe 38 and the liquid product is taken overhead in line 26. This liquid product in a line 26, together with the combined isobutylene-containing stream 44 (containing isobutylene from stream 22 from catalyst separator 4 and isobutylene-containing stream 42 from catalyst separator 40) is fractionated in a distillation tower 24 to yield a C₄ overhead 28 and a monoester product bottoms 30. In the case where the isobutylene feed stream is essentially pure, this overhead will be a recycle to a single isobutylene adsorption reactor. If the feed is a BB stream, a separate isobutylene adsorption reactor may be employed to accomplish maximum utilization of the available isobutylene.
It is unlikely that a single stage esterification reactor will accomplish the desirable degree of fatty acid conversion (ca 100%). Accordingly two or more reactors may be employed in series. Further to accomplish the last bit of fatty acid conversion it may be desirable to employ a technique for final conversion, such as the method taught by Jeromin et al. (U.S. Pat. No. 4,698,186) for reducing free fatty acids by esterifying with a lower monoalcohol in the presence of an acidic cation exchange resin as a solid esterification catalyst. As a final step it may be desirable to employ a caustic and water wash system to insure elimination of any significant amounts of free fatty acid in the final product.

4. Transesterification

Monoesters for biodiesel commonly are produced by a catalyzed reaction of a monoalcohol (typically methanol) with the triglycerides contained in vegetable and/or animal fats and oils. The catalyst system can be homogeneous or heterogeneous as described below.

a. Homogeneous Catalyzed Transesterification
The catalyst used in homogeneous systems is typically any base, most preferably sodium hydroxide or potassium hydroxide dissolved in the alcohol. The alcohol/catalyst mix then is charged into a closed reaction vessel and the oil or fat is added. Excess alcohol normally is used to ensure total conversion of the fat or oil to its esters. Care must be taken to monitor the amount of water and free fatty acids in the incoming oil or fat. If the free fatty acid level or water level is too high it may cause problems with soap formation and the separation of the glycerin/glycerol by-product downstream. The general biodiesel reaction is shown below: 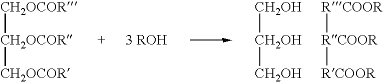
wherein R′, R″ and R′″ independently are the same or different fatty acid chains associated with the oil or fat, typically, but not limited to, palmitic, stearic, oleic and linolelic acids for naturally occurring oils and fats. R is any hydrocarbyl compound, generally an alkyl group, such as, but not limited to methyl, ethyl or a higher molecular weight alkyl group.
Once the reaction is complete, two major products exit: a glycerol-containing by-product and a monoester stream (biodiesel). Each has a substantial amount of the excess methanol that was used in the reaction. The glycerol phase is much denser than the biodiesel phase and the two can be gravity separated with glycerol simply drawn off the bottom of the settling vessel. Once the glycerol and biodiesel phases have been separated, the excess alcohol in each phase is removed for recovery and/or recycling. The glycerol phase also contains the catalyst and some organic compounds. The catalyst is neutralized and the organic compounds typically are separated by acidification.

b. Heterogeneous Catalyzed Transesterification
At least one process, the “Esterfip-H” process offered by Axens, is being commercialized using a heterogeneous catalyst for transesterification. A patent held by Axens' affiliate, Institute Francais du Petrole (Stern et al. U.S. Pat. No. 5,908,946), teaches the use of a catalyst employing oxides of zinc and aluminum. The process is reported to employ two stages of reactor and to achieve 99+% purity of methyl esters and provide yields close to 100%. Also, the glycerin produced is reported to be 98+% pure. A 50 million-gallon per year commercial plant is being built for Diester Industrie at Sete, France.
   5. Glycerin Etherization

There is considerable interest in developing a diesel fuel use for the by-product glycerin derived from the production of biodiesel. Gupta (U.S. Pat. No. 5,476,971 assigned to ARCO) teaches production of glycerin di-t-butyl ether in a liquid phase reaction of glycerin and isobutylene employing an acid catalyst (e.g., p-toluene-sulfonic acid). Kesling et al. (U.S. Pat. No. 5,308,365) teaches the benefits of dialkyl and tri-alkyl derivatives of glycerol in diesel fuel to reduce particle emissions. Further, Spooner-Wyman of Procter & Gamble (4^{th} AOCS Annual Meeting May 4-7, 2003—IOP5 Alternate Fuels) reports that “Di-butoxy glycerol is a promising candidate for blending in diesel fuel . . . ” In addition, the glycerol ethers can be valuable cloud point reducing additions to biodiesel.

6. Petroleum Refining Processes

In addition to the above processes relating to the production of biodiesel from vegetable and animal fats and oils, there are several conventional refining processes that enter into consideration of the benefits of integrating biodiesel production into a hydrocarbon processing operation such as petroleum refining and petrochemical production. These processes are summarized below, but are well described in the patent literature.

a. Olefin Producing Processes
Olefin containing streams generally result from cracking operations. For example, if a paraffin is cracked into two molecules, one of the resulting fragments will be an olefin. Typical cracking operations include the following:
i. Catalytic Cracking
   This process is the core technology enabling a refinery to maximize production of transportation fuels from crude oil by “cracking” the fractions boiling above the range of diesel fuel into lighter components. While refiners try to maximize liquid (gasoline and light distillate) production from these operations, inevitably there is production of lighter fragments including olefins containing 2-4 carbon atoms. The C₄ fraction is particularly interesting in that due to the catalytic nature of the cracking process there is considerable production of isobutane and isobutylene.
ii. Deep Catalytic Cracking
   In recent years processes have been developed that employ the catalytic cracking concept with specific catalysts and operating conditions that maximize the yields of light olefins. For example, a development of RIPP of Beijing, China, commonly known as the DCC Process, which has been commercialized in China and in Thailand, produces olefins such as propylene for polymer production. In this case there is considerable co-production of C₄ and heavier olefins. Basis feed, one commercial plant of this design has produced 5.3 wt % ethylene, 18.5 wt % propylene and 13.3 wt % butylenes.
iii. Thermal Cracking
   While less prevalent than catalytic cracking in the refinery, there is still substantial use of thermal processes such as coking and visbreaking in the processing of the heavier fractions of crude oil. These processes also yield substantial quantities of light olefins.
   On the petrochemical side however, thermal cracking—or pyrolysis—is the core technology used in converting various hydrocarbon fractions into light olefins and aromatic fractions for production of polymers and other petrochemical products. Economic utilization of the C₄ and heavier olefins is often a critical element in the overall economics of the operation.

Table 2 presents typical analysis of some light olefin streams from catalytic cracking and pyrolysis.

TABLE 2

Catalytic Cracking

| Composition - mol % | C4 cut | C5 cut | Pyrolysis C₄ |
|---|---|---|---|
| isobutane | 35 | | 3 |
| n-butane | 11 | | 3 |
| isobutylene | 15 | | 23 |
| n-butylene | 39 | | 22 |
| butadiene | <0.2 | | 49 |
| iso pentane | | 40 | |
| n-pentane | | 25 | |
| isoamylene | | 24 | |
| other amylenes | | 11 | |

b. Olefin Utilization Processes
The petroleum refining and petrochemical industries have developed several processes for adding value to the light olefin by-products from their operations. These are summarized below, but are well reported in the patent literature.
i. Alkylation
   Most refineries employing catalytic cracking also employ alkylation of C₃-C₅ olefins with isobutane to yield a superior gasoline blending stock. As the isobutane/olefin consumption ration is essentially equal molar, and while considerable isobutane is produced in catalytic cracking, it is generally insufficient to convert even the butylenes, let alone the propylene produced in the cat cracker.
ii. Oligomerization
   Often alkylation is combined with polymerization processes that produce dimers, or higher oligomers, that can be blended with gasoline or with diesel fuel. The traditional technology has employed a phosphoric acid catalyst supported on a matrix such as kieselguhr or silica. More recent developments include the Dimersol process of IFP that employs a homogeneous Ziegler-type catalyst system, which enables production of gasoline fractions from C₂-C₄ olefins from cat cracking or pyrolysis.
   Of perhaps more relevance to the biodiesel picture is the Polynaphtha Process—also of IFP. This process uses a solid catalyst system and converts C₃ and C₄ olefins into higher olefins for blending into gasoline, jet fuel and/or diesel fuel. After hydrotreating, the diesel fuel from this process is reported to have a cetane number of 40-45 and a superior pour point (−60° C.).

Employing combinations of the processes described above, the inventors have conceived several preferred embodiments. In the following descriptions of preferred embodiments, the various process steps are referred to the corresponding descriptions using the numbers employed above (e.g triglyceride hydrolysis is referred to as (1)).

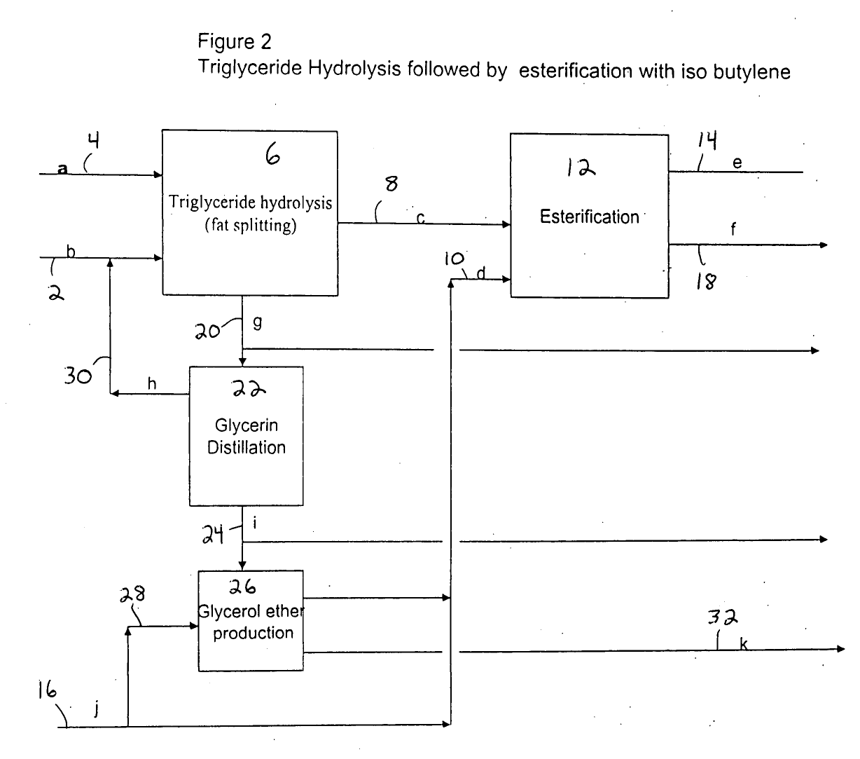

A. Reaction of Fatty Acids with Isobutylene (FIG. 2)

This process operation involves reacting with water from a line 2 (hydrolysis) of triglycerides (per 1) contained in a oil/fat stream 4 in a triglyceride hydrolysis reactor 6 to produce fatty acids 8, which then are reacted with isobutylene from an isobutylene-containing stream 10 in an esterification reactor 12 per (per 3b) to produce monoester product 14. The isobutylene-containing stream 10 is derived from a stream 16 from one or more of the olefin producing processes (per 6a). The solid acid based esterification reaction in reactor 12 preferentially will involve isobutylene and the unconverted butylenes and butanes are withdrawn in a line 18 for feeding to other processes, blended into gasoline or sold as a component of LPG.

In some cases it may be more advantageous to employ relatively pure isobutylene in the esterification reaction and possibly employ a recycle stream of isobutylene to maintain a sufficient concentration of isobutylene to accomplish the desired degree of esterification. Relatively pure isobutylene for this purpose can be separated from a butane/butylene stream by methods known in the art. One such method involves reaction of isobutylene with methanol to form MTBE, with subsequent cracking of the MTBE to produce isobutylene and methanol.

By-product glycerin in a line 20 produced from the hydrolysis reactor 6 can be sold or further processed offsite, or optionally concentrated by water removal in a glycerin distillation unit 22 to yield a relatively pure glycerin stream 24, which then can be used to produce glycerol di or tri t-butyl ether in a glycerol ether production unit 26 (per 5) by reaction with isobutylene in a line 28 obtained as a draw from isobutylene feed 16. The overhead from glycerin distillation 22 can be removed in a stream 30 and recycled to the hydrolysis step. The glycerol di or tri t-butyl ether is removed from unit 26 in a line 32 and can be blended into the diesel fuel product.

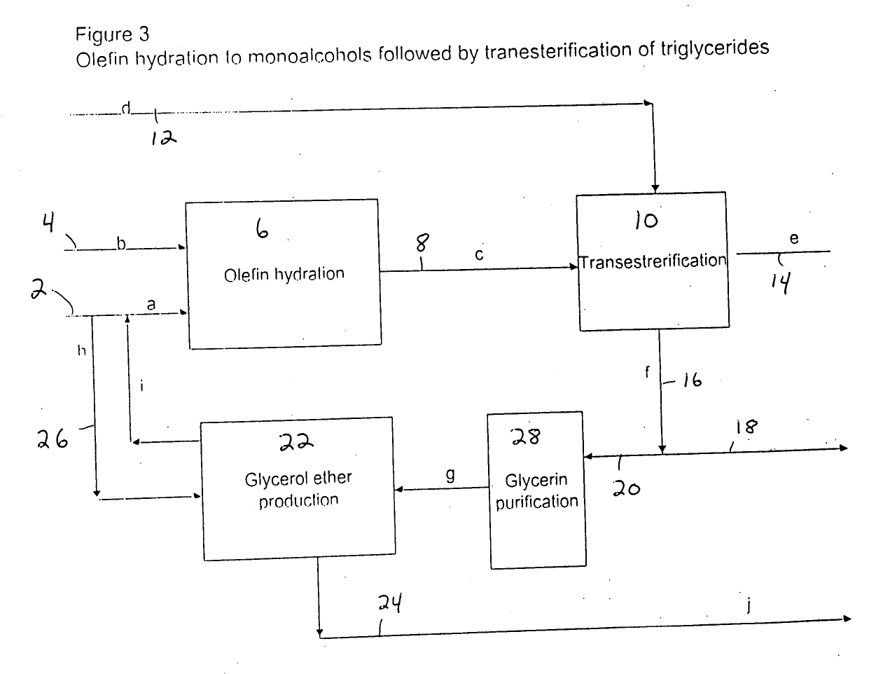

B. Transesterification of Triglyceride Containing Streams with Monoalcohols Derived from C₂ and Higher Olefins (FIG. 3)

C₂-C₅ Olefins in a line 2 (derived from 6a) and water in a line 4 first are converted in an olefin hydration reactor 6 to alcohols in a line 8 per (2). These alcohols 8 then are employed in a transesterification reaction in a reactor 10 per (4a or 4b) with triglyceride containing animal or vegetable oils or fats from a feed line 12 to produce monoester product in a line 14.

The glycerin produced as a by product in a line 16 from the transesterification reactor 10 can be taken as a product for sale in a line 18 for further processing, or may be taken in a line 20 and reacted with a draw line 26 from olefin feed line 2 in a glycerol ether reactor 22 to produce glycerol t-butyl ether in a line 24 per (5) either directly or after purification in unit 28.

C. Reaction (per 3a) of Alcohols Produced from Olefins (per 2) with Fatty Acids Produced by Hydrolysis (per 1) to Produce Monoesters and By-product Water

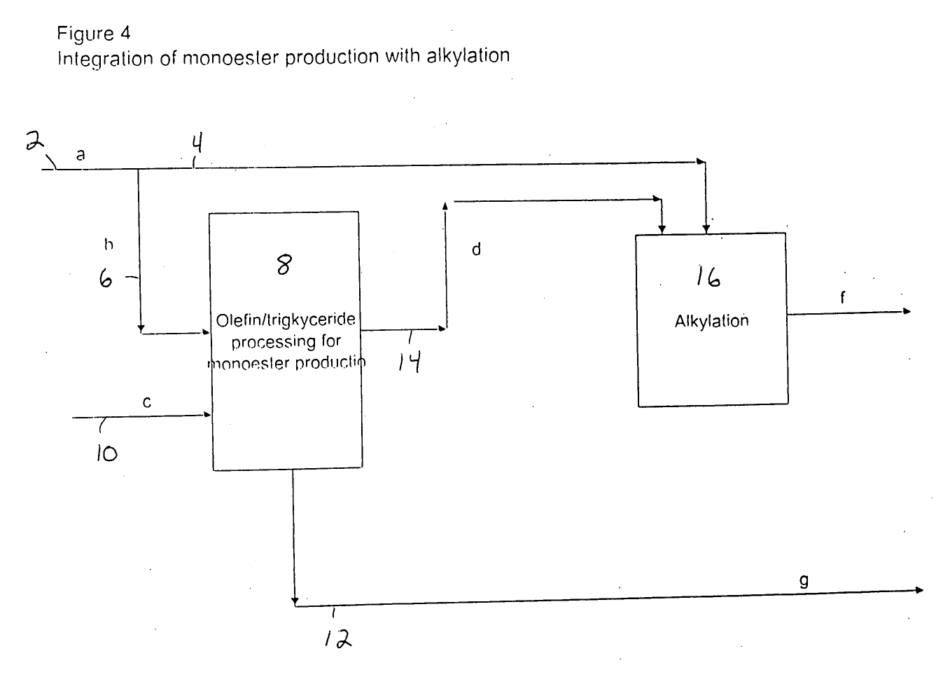

D. Integration of Monoester Production with Alkylation (FIG. 4)

Alkylation (per 6bi above) involves essentially equal molar quantities of isobutane and olefins. Inspection of the C₄ cut from catalytic cracking shows a deficiency of isobutane (35 mol % isobutane and 54 mol % butylenes). Additional isobutane is generally available as a by-products from other refinery processes, such as, but not limited to, catalytic reforming of naphtha, but in most cases there will at best be barely enough isobutane to convert all of the butylenes available. To bring the isobutane/olefin ratio closer to balance, the processes described in A and B above (FIGS. 2 & 3) can be employed as shown in FIG. 4. An olefin containing stream 2 is divided into a stream 4 going directly to alkylation and a stream 6, which is diverted to the production of monoesters in unit 8. Monoester production unit 8 involves reaction of triglyceride containing stream(s) 10 with the olefins in stream 6 to produce monoesters in a stream 12 and a by-product glycerin stream 14. After the olefins required for monoester production have been removed in a stream 14, the unconverted olefins and paraffins are sent to the alkylation unit 16 together with the olefins from stream 4 that by-passed the monoester production step.

E. Integration of Monoester Production with Olefin Oligomerization

Monoester production per either A or B above can be integrated with oligomerization of olefins (per 6bii). In the specific case of the Polynaphtha Process, the properties of the products complement each other in a fashion leading to a superior diesel product. The Polynaphtha product is reported to have a rather low cetane number (40-45), but a superior pour point. On the other hand as reported by Knoethe (Fuel vol 82 (2003), 971-975) the cetane numbers of fatty acid esters are quite high:

FIG. 3 - Cetane numbers of fatty acid esters

| Ester | Palmitic acid | Stearic acid | Oleic acid |
|---|---|---|---|
| Ethyl | 93 | 98 | 68 |
| Propyl | 85 | 91 | 59 |
| isobutyl | 84 | 99 | 60 |

From the foregoing descriptions, one skilled in the art can easily ascertain the essential characteristics of this invention, and without departing from the spirit and scope thereof, can make various obvious changes and modifications of processing details of this invention to adopt it to various usages and conditions.

1. A process for producing monoesters from a triglyceride-containing stream comprising vegetable or animal fats and/or oils, said process comprising either (I) a transesterification process or a (II) fatty acid olefin reaction process;
wherein said (I) transesterification process comprises the following steps:
(a) hydrolyzing olefins in an olefin containing stream comprising at least one olefin selected from the group consisting of ethylene, propylene, butylenes, amylene, hexane and mixtures thereof, said olefin-containing stream being derived from a petroleum or natural gas liquids process plant, whereby said olefins are converted to the corresponding alcohol;
(b) reacting said corresponding alcohol with triglyceride in said triglyceride containing streamin the presence of either a homogeneous or heterogeneous catalyst to produce monoesters and glycerin; and
(c) separating the monoesters from said glycerin and optionally purifying said monoesters; or
wherein said (II) fatty acid olefin reaction process comprises:
(i) hydrolyzing said triglyceride-containing stream to produce fatty acids and glycerins; and
(ii) reacting said fatty acids with olefins in an olefin containing stream comprising at least one olefin selected from the group consisting of ethylene, propylene, butylenes, amylene, hexane and mixtures thereof, said olefin-containing stream being derived from a petroleum or natural gas liquids process plant in the presence of a catalyst to produce monoesters.

2. A process as defined in claim 1 wherein said monoesters subsequently are employed as biodiesel.

3. A process as defined in claim 1 wherein said homogeneous catalyst comprises an alkali or an acid.

4. A process as defined in claim 3 wherein said alkali catalyst comprises sodium hydroxide, potassium hydroxide or mixtures thereof.

5. A process as defined in claim 3 wherein said separated glycerin in step (I)(c) comprises alkali catalyst and said process further comprises the step of neutralizing said alkali catalyst with an inorganic or organic acid.

6. A process as defined in claim 5 wherein said inorganic or organic acids are selected from the group consisting of sulfuric acid, hydrochloric acid, acetic acid, lactic acid and mixtures thereof.

7. A process as defined in claim 1 wherein said catalyst in step (II)(ii) comprises an acidic ion exchange resin catalyst.

8. A process as defining in claim 7 wherein said olefin-containing stream comprises isobutylene.

9. A process as defined in claim 8 wherein said isobutylene is obtained as a relatively pure stream by separation from said olefin-containing stream.

10. A process as defined in claim 7 wherein said catalyzed reaction step (II)(ii) comprises the following two steps: (1) contacting said olefin-containing stream with said acidic ion exchange resin catalyst to form carbonium ions on the catalyst's acidic sites based on isobutylene and (2) contacting said carbonium ions formed on the catalyst acidic sites with fatty acids to produce said monoesters.

11. A process as defined in claim 1 further comprising reacting glycerin produced during said process with isobutylene to produce di- and tri- glycerol ethers.

12. A process as defined in claim 1 wherein said process is integrated with an alkylation process in a petroleum refining plant, said integration comprising balancing the conversion of olefins in the olefin-containing stream with isobutene availability for the alkylation process.
